(19) United States
(12) Patent Application Publication
(10) Pub. No.: US 2025/0272834 A1
(43) Pub. Date: Aug. 28, 2025

(54) ECHOCARDIOGRAM ARTIFICIAL INTELLIGENCE PROCESSING SYSTEM, ECHOCARDIOGRAM ARTIFICIAL INTELLIGENCE PROCESSING METHOD AND COMPUTER PROGRAME PRODUCT

(71) Applicant: China Medical University, Taichung City (TW)

(72) Inventors: Kai-Sheng HSIEH, Kaohsiung City (TW); Chih-Chung HUANG, Tainan City (TW); Yu-Lung HSU, Taichung City (TW); Shih-Sheng CHANG, Taichung City (TW)

(21) Appl. No.: 19/059,238

(22) Filed: Feb. 20, 2025

(30) Foreign Application Priority Data
Feb. 23, 2024 (TW) 113106679

(51) Int. Cl. G06T 7/00; A61B 8/08; G06V 10/62; G06V 10/764; G06V 20/40

(52) U.S. Cl. CPC G06T 7/0012; A61B 8/0883; G06V 10/62; G06V 10/764; G06V 20/49; G06T 2207/10016; G06T 2207/10132; G06T 2207/20056; G06T 2207/30048; G06V 2201/031

(57) ABSTRACT

An echocardiogram artificial intelligence processing system includes an ultrasound detecting device and a processor. The ultrasound detecting device obtains an echocardiogram video. A splitting module splits the echocardiogram video into a plurality of frames. Each frame includes a plurality of position points. A gray value obtaining module is to obtain a gray value of each of the position points of each of the frames, and to collect the gray value corresponding to an identical one of the position points of each frame to form a gray value variation chart corresponding to the identical one of the position points. A frequency chart converting module converts the gray value variation chart corresponding to each position point into a frequency chart. A frequency averaging module obtains an echocardiogram variation feature chart. An artificial intelligence classifying module is signally connected to the frequency averaging module.

### RELATED APPLICATIONS

This application claims priority to Taiwan Application Serial Number 113106679, filed Feb. 23, 2024, which is herein incorporated by reference.

### BACKGROUND

### Technical Field

The present disclosure relates to an artificial intelligence processing system, an artificial intelligence processing method and a computer program product. More particularly, the present disclosure relates to an echocardiogram artificial intelligence processing system, an echocardiogram artificial intelligence processing method and a computer program product.

### Description of Related Art

Recently, artificial intelligence techniques are developing quickly, and are applied to fields such as vehicles and medicine. In the medicine filed, initial analysis and classification for the obtained data may be conducted by the artificial intelligence technique, which may be served as assisting data for the professional physicians to judge.

Deep learning methods are currently used in echocardiogram, a deep learning model is trained by relative samples, and after new detected echocardiogram image is input to the deep learning model, the size of the heart may be marked automatically, and the structure and shape of the heart may be defined. However, the method is to use static images to identify structure and shape of the heart, but the frequency variation of the heart rate in the echocardiogram image is not considered, and there is a space for improvement.

### SUMMARY

An echocardiogram artificial intelligence processing system is provided according to one embodiment of the present discourse, which includes an ultrasound detecting device and a processor. The ultrasound detecting device is configured to touch a to-be-detected body to obtain an echocardiogram video. The processor is signally connected to the ultrasound detecting device and includes a video splitting module, a gray value obtaining module, a frequency chart converting module, a frequency chart averaging module and an artificial intelligence classifying module. The video splitting module is configured to split the echocardiogram video into a plurality of frames. The gray value obtaining module is signally connected to the video splitting module, each of the frames includes a plurality of position points, and the gray value obtaining module is configured to obtain a gray value corresponding to each of the position points of each of the frames, and to collect the gray value of an identical one of the position points corresponding to each of the frames to form a gray value variation chart corresponding to the identical one of the position points. The frequency chart converting module is signally connected to the gray value obtaining module, and converts the gray value variation chart corresponding to each of the position points into a frequency chart. The frequency chart averaging module is signally connected to the frequency chart converting module, and averages the frequency charts corresponding to the position points, thereby obtaining an echocardiogram variation feature chart. The artificial intelligence classifying module is signally connected to the frequency chart averaging module, and uses a classifying model to judge whether the echocardiogram variation feature chart belongs to a first class or a second class.

An echocardiogram artificial intelligence processing method is provided according to another embodiment of the present discourse, which includes an echocardiogram video obtaining step, a video splitting step, a gray value obtaining step, a frequency chart converting step, a frequency chart averaging step and an artificial intelligence classifying step. In the echocardiogram video obtaining step, an ultrasound detecting device touches a to-be-detected body to obtain an echocardiogram video. In the video splitting step, a video splitting module of an echocardiogram artificial intelligence processing system is used, and splits the echocardiogram video into a plurality of frames. In the gray value obtaining step, each of the frames comprises a plurality of position points, and a gray value obtaining module of the echocardiogram artificial intelligence processing system is used, obtains a gray value of each of the position points of each of the frames, and collects the gray value corresponding to an identical one of the position points of each of the frames to form a gray value variation chart corresponding to the identical one of the position points. In the frequency chart converting step, a frequency chart converting module of the echocardiogram artificial intelligence processing system is used, and converts the gray value variation chart of each of the position points into a frequency chart. In the frequency chart averaging step, a frequency chart averaging module of the echocardiogram artificial intelligence processing system is used, and averages the frequency charts corresponding to the position points, thereby obtaining an echocardiogram variation feature chart. In the artificial intelligence classifying step, a classifying model of an artificial intelligence classifying module of the echocardiogram artificial intelligence processing system is used, and judges whether the echocardiogram variation feature chart belongs to a first class or a second class.

A computer program product is provided according to still another embodiment of the present discourse, being stored in a machine readable medium and including at least one instruction, the at least one instruction is performed by one or more computers, and the one or more computers preform: obtaining an echocardiogram video; splitting the echocardiogram video into a plurality of frames; each of the frames including a plurality of position points, obtaining a gray value of each of the position points of each of the frames, collecting the gray value of an identical one of the position points of each of the frames to form a gray value variation chart corresponding to the identical one of the position points; converting the gray value variation chart of each of the position points into a frequency chart; averaging the frequency charts corresponding to the position points, thereby obtaining an echocardiogram variation feature chart; and judging whether the echocardiogram variation feature chart belongs to a first class or a second class.

### BRIEF DESCRIPTION OF THE DRAWINGS

The present disclosure can be more fully understood by reading the following detailed description of the embodiment, with reference made to the accompanying drawings as follows:

FIG. 1 shows a system block chart of an echocardiogram artificial intelligence processing system according to one embodiment of the present disclosure.

FIG. 2A shows one frame of the echocardiogram artificial intelligence processing system of the embodiment of FIG. 1.

FIG. 2B shows another frame of the echocardiogram artificial intelligence processing system of the embodiment of FIG. 1.

FIG. 2C shows still another frame of the echocardiogram artificial intelligence processing system of the embodiment of FIG. 1.

FIG. 3A shows a plurality of position points of the frame of the embodiment of FIG. 2A.

FIG. 3B shows a plurality of position points of the frame of the embodiment of FIG. 2B.

FIG. 3C shows a plurality of position points of the frame of the embodiment of FIG. 2C.

FIG. 4A shows gray values of a position point array of the embodiment of FIG. 3A.

FIG. 4B shows gray values of a position point array of the embodiment of FIG. 3B.

FIG. 4C shows gray values of a position point array of the embodiment of FIG. 3C.

FIG. 5A shows a gray value variation chart of one position point of the embodiment of FIGS. 3A to 3C.

FIG. 5B shows a gray value variation chart of another position point of the embodiment of FIGS. 3A to 3C.

FIG. 5C shows a gray value variation chart of still another position point of the embodiment of FIGS. 3A to 3C.

FIG. 6 shows an echocardiogram variation feature chart of the embodiment of FIGS. 3A to 3C.

FIG. 7 shows a block flow chart of an echocardiogram artificial intelligence processing method according to another embodiment of the present disclosure.

### DETAILED DESCRIPTION

The embodiments of the present disclosure will be illustrated with drawings hereinafter. In order to clearly describe the content, many practical details will be mentioned with the description hereinafter. However, it will be understood by the reader that the practical details will not limit the present disclosure. In other words, in some embodiments of the present disclosure, the practical details are not necessary. Additionally, in order to simplify the drawings, some conventional structures and elements may be illustrated in the drawings in a simple way; the repeated elements may be labeled by the same or similar reference numerals.

In addition, the terms first, second, third, etc. are used herein to describe various elements or components, these elements or components should not be limited by these terms. Consequently, a first element or component discussed below could be termed a second element or component. Moreover, the combinations of the elements, the components, the mechanisms and the modules are not well-known, ordinary or conventional combinations, and whether the combinations can be easily completed by the one skilled in the art cannot be judged based on whether the elements, the components, the mechanisms or the module themselves are well-known, ordinary or conventional.

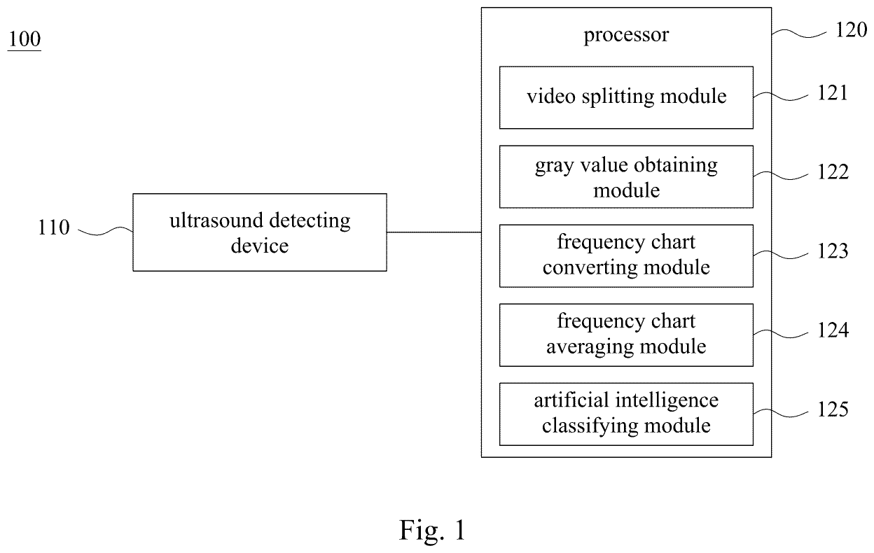

Please refer to FIG. 1, and FIG. 1 shows a system block chart of an echocardiogram artificial intelligence processing system 100 according to one embodiment of the present disclosure. The echocardiogram artificial intelligence processing system 100 includes an ultrasound detecting device 110 and a processor 120. The ultrasound detecting device 110 is configured to touch a to-be-detected body to obtain an echocardiogram video. The ultrasound detecting device 110 may use a high frequency sound wave to pass through the to-be-detected body, and with different reflection of different tissues, the structures of the tissues inside the to-be-detected body may be shown. Hence, as the ultrasound detecting device 110 touches a portion corresponding to a heart of the to-be-detected body, the echocardiogram video may be obtained, which is a dynamic video. Moreover, if the echocardiogram video includes personal information of the to-be-detected body, the personal information may be removed, and further processing is conducted.

The processor 120 is signally connected to the ultrasound detecting device 110 and includes a video splitting module 121, a gray value obtaining module 122, a frequency chart converting module 123, a frequency chart averaging module 124 and an artificial intelligence classifying module 125. The processor 120 may be a center processing unit of a computer, and the computer may further include a main memory such as a dynamic random-access memory, and a compact disc driving portion reading data from the media such as compact discs. In addition, the processor 120 may be programmable to form the video splitting module 121, the gray value obtaining module 122, the frequency chart converting module 123, the frequency chart averaging module 124 and the artificial intelligence classifying module 125 to conduct corresponding instructions. In other embodiments, the processor may be a processor of other electronic devices having calculating functions.

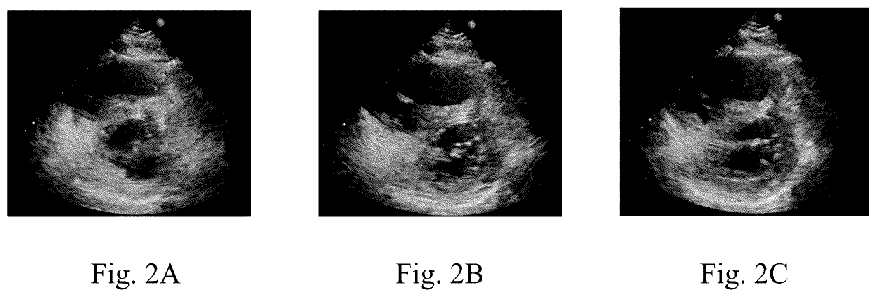

Please refer to FIG. 2A to FIG. 2C, with reference of FIG. 1, FIG. 2A shows one frame of the echocardiogram artificial intelligence processing system 100 of the embodiment of FIG. 1, FIG. 2B shows another frame of the echocardiogram artificial intelligence processing system 100 of the embodiment of FIG. 1, and FIG. 2C shows still another frame of the echocardiogram artificial intelligence processing system 100 of the embodiment of FIG. 1. After the ultrasound detecting device 110 obtains the echocardiogram video, the video splitting module 121 may split the echocardiogram video into a plurality of frames, for example splitting by seconds, the frames respectively correspond to the 1st second, the 2nd second and so on are obtained, and three continuous frames are shown in FIG. 2A to FIG. 2C.

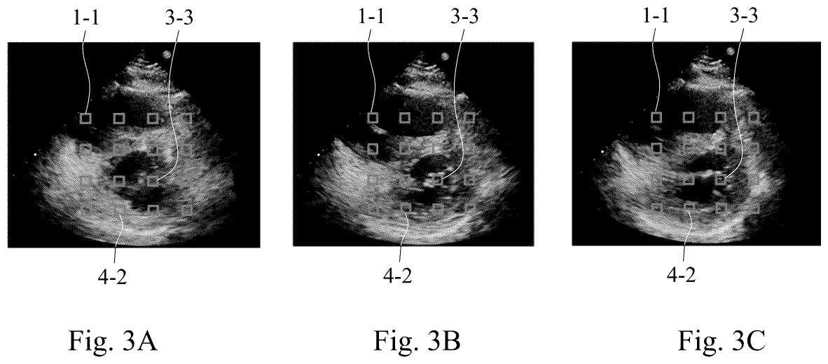

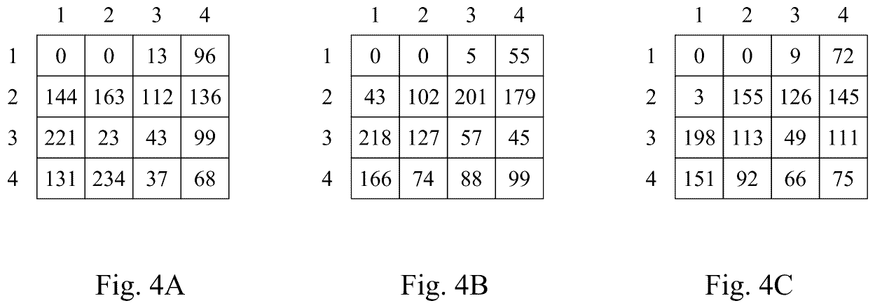

Please refer to FIG. 3A to FIG. 3C and FIG. 4A to FIG. 4C, with reference of FIG. 1, FIG. 3A shows a plurality of position points of the frame of the embodiment of FIG. 2A, FIG. 3B shows a plurality of position points of the frame of the embodiment of FIG. 2B, FIG. 3C shows a plurality of position points of the frame of the embodiment of FIG. 2C, FIG. 4A shows gray values of a position point array of the embodiment of FIG. 3A, FIG. 4B shows gray values of a position point array of the embodiment of FIG. 3B, and FIG. 4C shows gray values of a position point array of the embodiment of FIG. 3C. The gray value obtaining module 122 is signally connected to the video splitting module 121, and after splitting the echocardiogram video into the plurality of frames, each of the frames may include a plurality of position points, and the gray value obtaining module 122 is configured to obtain a gray value of each of the position points of each of the frames.

Precisely, the position points of each of the frames may form an N×N position point array, and N is an integer. As shown in FIG. 3A to FIG. 3C, the position points of each of the frames may form a 4×4 position point array, and sixteen position points may be included. In order to clarify the drawings, only the position point 1-1 of the first column and the first row, the position point 3-3 of the third column and the third row and the position point 4-2 of the fourth column and the second row are marked. Moreover, the gray values of each of the position points of FIG. 3A are shown in FIG. 4A, the gray values of each of the position points of FIG. 3B are shown in FIG. 4B, and the gray values of each of the position points of FIG. 3C are shown in FIG. 4C. Additionally, although sixteen position points are shown in FIGS. 3A to 3C and form a rectangular position point array, in other embodiments, a number and locations of the position points may be adjusted according to the demands, there is no need to form a rectangular position point array, and the disclosure is not limited to the drawings.

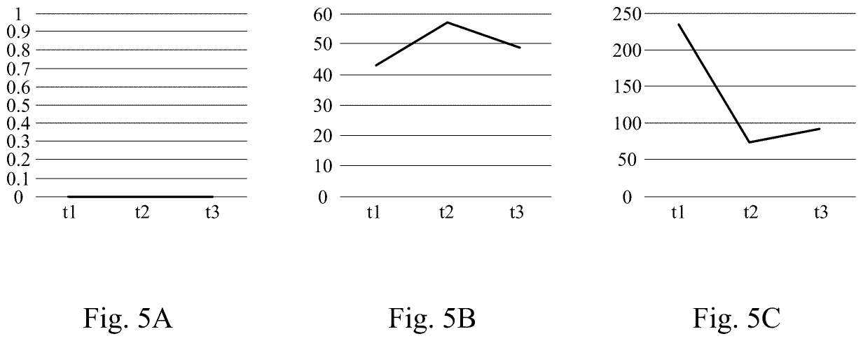

Please refer to FIG. 5A to FIG. 50, with references of FIG. 1 and FIG. 4A to FIG. 4C, FIG. 5A shows a gray value variation chart of one position point of the embodiment of FIGS. 3A to 3C, FIG. 5B shows a gray value variation chart of another position point of the embodiment of FIGS. 3A to 3C, and FIG. 5C shows a gray value variation chart of still another position point of the embodiment of FIGS. 3A to 3C. After obtaining the gray values of each of the position points of each of the frames, the gray value obtaining module 122 may collect the gray value of an identical one of the position points of each of the frames to form a gray value variation chart corresponding to the identical one of the position points. The gray values of the position point 1-1 in FIG. 4A to FIG. 4C are 0, 0, and 0, respectively, and the gray value variation chart formed therefrom is shown as FIG. 5A; the gray values of the position point 3-3 in FIG. 4A to FIG. 4C are 43, 57, and 49, respectively, and the gray value variation chart formed therefrom is shown as FIG. 5B; the gray values of the position point 4-2 in FIG. 4A to FIG. 4C are 234, 74, and 92, respectively, and the gray value variation chart formed therefrom is shown as FIG. 5C. Hence, sixteen gray value variation charts may be obtained.

The frequency chart converting module 123 is signally connected to the gray value obtaining module 122. After obtaining the gray value variation charts, the frequency chart converting module 123 converts the gray value variation chart of each of the position points into a frequency chart. Specially, a fast Fourier transform is performed to convert the gray value variation chart of each of the position points into the frequency chart.

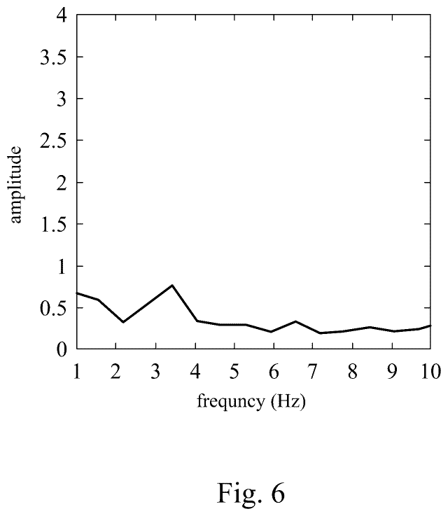

Please refer to FIG. 6, with reference of FIG. 1, and FIG. 6 shows an echocardiogram variation feature chart of the embodiment of FIGS. 3A to 3C. The frequency chart averaging module 124 is signally connected to the frequency chart converting module 123, and, after obtaining the sixteen frequency charts, all the frequency charts corresponding to all position points may be averaged, thereby obtaining an echocardiogram variation feature chart. Therefore, the whole echocardiogram video may be processed to convert into a single image, i.e., the echocardiogram variation feature char.

As shown in FIG. 1 and FIG. 6, the artificial intelligence classifying module 125 is signally connected to the frequency chart averaging module 124, and after obtaining the echocardiogram variation feature char, a classifying model may be used to judge whether the echocardiogram variation feature chart belongs to a first class or a second class. Specifically, the classifying model may be a YOLO model, during training, professional persons such as cardiologists may classify a plurality echocardiogram variation feature charts processed by the aforementioned method, thereby obtaining first echocardiogram variation feature charts belonging to the first class and second echocardiogram variation feature charts belonging to the second class training, the first class may for example be normal, and the second class may for example be abnormal, but the present disclosure is not limited thereto. The first echocardiogram variation feature charts and the second echocardiogram variation feature charts may then be used to train the artificial intelligence classifying module 125, thereby establishing the classifying model, and the echocardiogram variation feature chart in FIG. 6 may be judged to belong to the first class or the second class. In other embodiments, more classes may be sets, not to be limited to the first class and second class, and the classifying model may be a neural network model, but the present disclosure is not limited thereto.

Hence, with splitting the dynamic echocardiogram video to obtain multiple position points, and to obtain the average of the frequency charts of the multiple position points, complex heart exercising deformation can be simplified, and the heart exercise can be described by two-dimension frequency, thereby increasing the classification accuracy. Moreover, the dynamic video may be changed to static images, the variation of the shape may be observed, and based on the chart of the frequency spectrum, the artificial intelligence learning quality may not be easily effected by large variation caused by the variation of personal hobbits as the clinician operating the device.

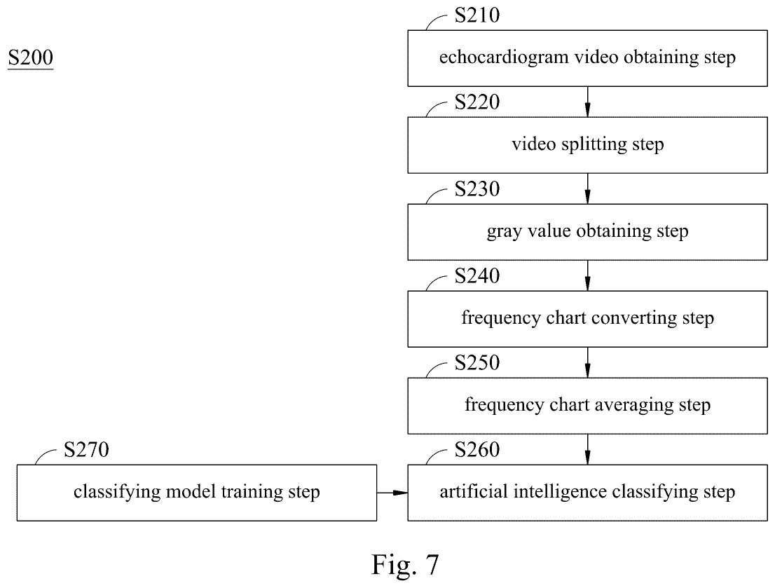

Please refer to FIG. 7, and FIG. 7 shows a block flow chart of an echocardiogram artificial intelligence processing method S200 according to another embodiment of the present disclosure. The echocardiogram artificial intelligence processing method S200 includes an echocardiogram video obtaining step S210, a video splitting step S220, a gray value obtaining step S230, a frequency chart converting step S240, a frequency chart averaging step S250 and an artificial intelligence classifying step S260. The echocardiogram artificial intelligence processing method S200 may be describe hereinafter with the echocardiogram artificial intelligence processing system 100 in FIG. 1 to FIG. 6.

In the echocardiogram video obtaining step S210, the ultrasound detecting device 110 touches the to-be-detected body to obtain the echocardiogram video.

In the video splitting step S220, the video splitting module 121 of the echocardiogram artificial intelligence processing system 100 is used, and splits the echocardiogram video into the plurality of frames.

In the gray value obtaining step S230, each of the frames includes a plurality of position points, and the gray value obtaining module 122 of the echocardiogram artificial intelligence processing system 100 is used, obtains the gray value of each of the position points of each of the frames, and collects the gray value of an identical one of the position points of each of the frames to form the gray value variation chart corresponding to the identical one of the position points.

In the frequency chart converting step S240, the frequency chart converting module 123 of the echocardiogram artificial intelligence processing system 100 is used, and converts the gray value variation chart of each of the position points into the frequency chart.

In the frequency chart averaging step S250, the frequency chart averaging module 124 of the echocardiogram artificial intelligence processing system 100 is used, and averages the frequency charts corresponding to the position points, thereby obtaining the echocardiogram variation feature chart.

In the artificial intelligence classifying step S260, the classifying model of the artificial intelligence classifying module 125 of the echocardiogram artificial intelligence processing system 100 is used, and judges whether the echocardiogram variation feature chart belongs to the first class or the second class.

Precisely, in the echocardiogram video obtaining step S210, professional persons may use the ultrasound detecting device 110 touch the to-be-detected body, and dynamic echocardiogram videos from different angles and different positions may be obtained.

In the video splitting step S220, the frames may be obtained by shoot the screen at predetermined time points, and the echocardiogram video may be split into the plurality of frames, as shown in FIG. 2A to FIG. 2C.

In the gray value obtaining step S230, the gray value obtaining module 122 may select the position points of each of the frames to form an N×N position point array, and N is an integer. As shown in FIG. 3A to FIG. 3C, N is four, a 4×4 position point array is formed, and the gray values obtained from each of the position points are shown in FIG. 4A to FIG. 4C. After which, the gray value corresponding to the same position point of each of the frames may be obtained, and form the gray value variation chart corresponding to the same position point, as shown in the gray value variation chart of the position point 1-1 in FIG. 5A, the gray value variation chart of the position point 3-3 in FIG. 5B and the gray value variation chart of the position point 4-2 in FIG. 5C.

In the frequency chart converting step S240, the fast Fourier transform is used by the frequency chart converting module 123 to convert the gray value variation chart of each of the position points into the frequency chart. In the frequency chart averaging step S250, the average of the sixteen frequency charts may be obtained, and the echocardiogram variation feature chart obtained therefrom is shown in FIG. 6.

Finally, the classifying model of the artificial intelligence classifying step S260 is a YOLO model, and the echocardiogram variation feature chart is classified by the classifying model, and is classified to the first class or the second class.

The echocardiogram artificial intelligence processing method S200 may further includes a classifying model training step S270, the plurality of first echocardiogram variation feature charts belonging to the first class and the plurality of second echocardiogram variation feature charts belonging to the second class are used to train the artificial intelligence classifying module 125, thereby establishing the classifying model. The first echocardiogram variation feature charts and the second echocardiogram variation feature charts are obtained from the echocardiogram video obtaining step S210, the video splitting step S220, the gray value obtaining step S230, the frequency chart converting step S240 and the frequency chart averaging step S250, but the first echocardiogram variation feature charts and the second echocardiogram variation feature charts are already judged to belong to the first class or the second class by professional persons in advance. Hence, the classifying model which can classify the first class and the second class may be established.

In still another embodiment, a computer program product is provided, which is stored in a machine readable medium and includes at least one instruction, the at least one instruction is performed by one or more computers, and the one or more computers preform: obtaining an echocardiogram video; splitting the echocardiogram video into a plurality of frames; each of the frames including a plurality of position points, obtaining a gray value of each of the position points of each of the frames, collecting the gray value of an identical one of the position points of each of the frames to form a gray value variation chart corresponding to the identical one of the position points; converting the gray value variation chart of each of the position points into a frequency chart; averaging the frequency charts corresponding to the position points, thereby obtaining an echocardiogram variation feature chart; and judging whether the echocardiogram variation feature chart belongs to the first class or the second class In other words, the echocardiogram video obtaining step, the video splitting step, the gray value obtaining step, the frequency chart converting step, the frequency chart averaging step and the artificial intelligence classifying step may be programed and stored in the disc, and the computer reads the disc, reads an input echocardiogram video for classification, and conducts relative instructions to classify the echocardiogram video.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An echocardiogram artificial intelligence processing system, comprising:
an ultrasound detecting device, configured to touch a to-be-detected body to obtain an echocardiogram video; and
a processor, signally connected to the ultrasound detecting device and comprising:
a video splitting module, configured to split the echocardiogram video into a plurality of frames;
a gray value obtaining module, signally connected to the video splitting module, wherein each of the frames comprises a plurality of position points, and the gray value obtaining module is configured to obtain a gray value corresponding to each of the position points of each of the frames, and to collect the gray value of an identical one of the position points corresponding to each of the frames to form a gray value variation chart corresponding to the identical one of the position points;
a frequency chart converting module, signally connected to the gray value obtaining module, and converting the gray value variation chart corresponding to each of the position points into a frequency chart;
a frequency chart averaging module, signally connected to the frequency chart converting module, and averaging the frequency charts corresponding to the position points, thereby obtaining an echocardiogram variation feature chart; and
an artificial intelligence classifying module, signally connected to the frequency chart averaging module, and using a classifying model to judge whether the echocardiogram variation feature chart belongs to a first class or a second class.

2. The echocardiogram artificial intelligence processing system of claim 1, wherein, the classifying model is a YOLO model.

3. The echocardiogram artificial intelligence processing system of claim 1, wherein, the frequency chart converting module performs a fast Fourier transform to convert the gray value variation chart corresponding to each of the position points into the frequency chart.

4. The echocardiogram artificial intelligence processing system of claim 1, wherein, the position points of each of the frames form an N×N position point array, and N is an integer.

5. An echocardiogram artificial intelligence processing method, comprising:
an echocardiogram video obtaining step, wherein an ultrasound detecting device touches a to-be-detected body to obtain an echocardiogram video;
a video splitting step, wherein a video splitting module of an echocardiogram artificial intelligence processing system is used, and splits the echocardiogram video into a plurality of frames;
a gray value obtaining step, wherein each of the frames comprises a plurality of position points, and a gray value obtaining module of the echocardiogram artificial intelligence processing system is used, obtains a gray value of each of the position points of each of the frames, and collects the gray value corresponding to an identical one of the position points of each of the frames to form a gray value variation chart corresponding to the identical one of the position points;
a frequency chart converting step, wherein a frequency chart converting module of the echocardiogram artificial intelligence processing system is used, and converts the gray value variation chart of each of the position points into a frequency chart;
a frequency chart averaging step, wherein a frequency chart averaging module of the echocardiogram artificial intelligence processing system is used, and averages the frequency charts corresponding to the position points, thereby obtaining an echocardiogram variation feature chart; and
an artificial intelligence classifying step, wherein a classifying model of an artificial intelligence classifying module of the echocardiogram artificial intelligence processing system is used, and judges whether the echocardiogram variation feature chart belongs to a first class or a second class.

6. The echocardiogram artificial intelligence processing method of claim 5, wherein, in the frequency chart converting step, the frequency chart converting module performs a fast Fourier transform to convert the gray value variation chart corresponding to each of the position points into the frequency chart.

7. The echocardiogram artificial intelligence processing method of claim 5, wherein, in the artificial intelligence classifying step, the classifying model is a YOLO model.

8. The echocardiogram artificial intelligence processing method of claim 5, wherein, in the gray value obtaining step, the gray value obtaining module selects the position points of each of the frames to form an N×N position point array, and N is an integer.

9. The echocardiogram artificial intelligence processing method of claim 5 further comprising a classifying model training step, wherein a plurality of first echocardiogram variation feature charts belonging to the first class and a plurality of second echocardiogram variation feature charts belonging to the second class are used to train the artificial intelligence classifying module, thereby establishing the classifying model.

10. A computer program product, being stored in a machine readable medium and comprising at least one instruction, the at least one instruction being performed by one or more computers, and the one or more computers preforming:
obtaining an echocardiogram video;
splitting the echocardiogram video into a plurality of frames;
each of the frames comprising a plurality of position points, obtaining a gray value of each of the position points of each of the frames, collecting the gray value of an identical one of the position points of each of the frames to form a gray value variation chart corresponding to the identical one of the position points;
converting the gray value variation chart of each of the position points into a frequency chart;
averaging the frequency charts corresponding to the position points, thereby obtaining an echocardiogram variation feature chart; and
judging whether the echocardiogram variation feature chart belongs to a first class or a second class.
